(19) United States
(12) Patent Application Publication
(10) Pub. No.: US 2009/0281263 A1
(43) Pub. Date: Nov. 12, 2009

(54) DEVICE AND METHOD FOR PRODUCING ACRYLIC ACID WITH A REDUCED AUTOXIDATION TENDENCY

(76) Inventors: Jürgen Mosler, Castrop-Rauxel (DE); Günther Bub, Marl (DE)

Correspondence Address: SMITH MOORE LEATHERWOOD LLP, P.O. BOX 21927, GREENSBORO, NC, 27420, US

(21) Appl. No.: 12/374,205

(22) PCT Filed: Jul. 20, 2007
(86) PCT No.: PCT/EP07/06479

§ 371 Date: Jan. 16, 2009

(30) Foreign Application Priority Data
Jul. 21, 2006 (DE) 10 2006 036 177.6

(51) Int. Cl. C08F 120/06; C07C 51/16; C07C 57/18; B01J 19/00

(52) U.S. Cl. 526/317.1; 562/532; 562/598; 422/189

(57) ABSTRACT

The present invention relates to a device for the preparation of acrylic acid, comprising a first reactor and at least one further reactor, wherein at least the first reactor is a multi-tube reactor having a plurality of tubes comprising a catalyst which catalyses the synthesis of acrolein, wherein the tubes open into a collection chamber which can be fluidically connected to the at least one further reactor via an outflow region, wherein the outflow region comprises a laminarization means causing laminarization of a flow profile of a gas flowing through the outflow region. The invention also relates to a process for the preparation of acrylic acid, to an acrylic acid, to a process for the preparation of a hydrophilic polymer, to a hydrophilic polymer, to a method for the production of a water-absorbent hygiene article, to chemical products, and to the use of an acrylic acid.

The present invention relates to a device and a process for the preparation of acrylic acid, to an acrylic acid, to a process for the preparation of a hydrophilic polymer, to a hydrophilic polymer, to a method for the production of a water-absorbent hygiene article, to chemical products such as fibers, shaped articles or films and also to the use of an acrylic acid.

Acrylic acid is the starting substance for a large number of polymers. In particular, acrylic acid is also the starting substance for what are known as superabsorbent polymers which are based on crosslinked, partially neutralized polyacrylates and are able to absorb more than ten times their own weight in water. Acrylic acid is often prepared from acrolein which, in turn, is produced by gas phase oxidation of propene. In this gas phase oxidation of propene, what is known as autoxidation is often a problem in which, instead of the propene being partially oxidized as desired so as to form acrolein, the propene is completely oxidized in an undesirable manner so as to form undesirable by-products.

In general terms, the object of the present invention was to overcome the drawbacks resulting from the prior art in conjunction with the preparation of acrylic acid by two-stage gas phase oxidation of propene.

In particular, the object of the present invention was to specify a process and a device for the preparation of acrylic acid from propene allowing the acrylic acid to be obtained in a yield which is as high as possible.

In particular, the object of the present invention was to propose a device and a process for the preparation of acrylic acid in which the autoxidation of acrolein is reduced compared to conventional reactors or conventional processes.

The subject-matters of the claims help to achieve the foregoing objects, the dependent claims presenting special embodiments of the invention.

The device according to the invention for the preparation of acrylic acid comprises
a first reactor and
at least one further reactor,
   wherein at least the first reactor is a multi-tube reactor having a plurality of tubes comprising a catalyst which catalyzes the synthesis of acrolein from propene,
      wherein the tubes open into a collection chamber which can be fluidically connected to the at least one further reactor via an outflow region,
      wherein the outflow region comprises a laminarization means causing laminarization of a flow profile of a gas flowing through the outflow region.

According to the invention, the term “fluidically” refers to the fact that gases or liquids, including suspensions, or the mixtures thereof, preferably gases, are guided through corresponding lines. Pipelines, pumps and the like may, in particular, be used for this purpose.

In one configuration of the present invention, the first and the second reactor can form portions of a large-scale reactor which receives the first and the subsequent second reactor. In a large-scale reactor of this type, both reaction stages are configured with respectively differing catalysts for the preparation of acrolein in the first partial reactor and the conversion of acrolein to acrylic acid in the second partial reactor.

In the present context, the term “laminarization of a flow profile” refers, in particular, to the reduction of the Reynolds number of the corresponding flow. A person skilled in the art is familiar with various methods for determining the Reynolds number. For example, an average Reynolds number may be determined by determining, on the one hand, the flow speed or the mass flow rate and, on the other hand, the viscosity of the gas flow. The flow speed and/or the mass flow rate can be determined using a conventional flowmeter, whereas the viscosity of the gas flow can be calculated, for example, based on the composition of the gas flow and the temperature. In principle, a calculation, especially an iterative calculation, can also be carried out based on the corresponding Navier-Stokes equations. Conventional continuous fluid dynamics (CFD) methods, for example the FLUENT™ software package, can, in particular, be used in this case.

Furthermore, the Reynolds number can be calculated using known time-of-flight (TOF) and/or marker methods in which, for example, a flow profile of the gas is measured and adaptation to this profile is carried out by varying the Reynolds number. In addition, the laminarization can also advantageously be determined based on a model of the device, for example based on a Reynolds colored thread test.

The term “multi-tube reactor” refers, in particular, to a reactor having a large number of mutually parallel tubes through which educt and product gases are able to flow. These tubes are often filled with a feedstock comprising at least one catalyst which is selected so as to catalyze a corresponding reaction. In the case of the first reactor, the catalyst is, in particular, selected so as to catalyze the synthesis of acrolein from propene. The catalysts contained may be any catalysts which are known to a person skilled in the art and are conventionally used in the gas phase oxidation of propene to form acrolein. In particular, the catalysts are generally oxidic multi-component systems conventionally based on molybdenum, chromium, vanadium or tellurium oxides. In relation to suitable catalysts in the preparation of acrolein from propene, reference is made to “Stets Geforscht”, Volume 2, Chemieforschung im Degussa-Forschungszentrum Wolfgang 1998, pp. 108-126, “Acrolein und Derivate” Chapter, Dietrich Amtz and Ewald Noll and, in particular, to WO 03/051809 A1, the disclosure of which is incorporated herein by reference.

It is particularly preferred for the catalyst contained in the first reactor to have the composition:

MoₐBi_{b}Fe_{c}A_{d}BₑC_{f}D_{g}Oₓ

in which

Mo represents molybdenum,

Bi represents bismuth,

Fe represents iron,

A represents at least one element selected from cobalt and nickel,

B represents at least one element selected from an alkali metal, an alkaline-earth metal and thallium,

C represents at least one element selected from tungsten, silicon, aluminium, zirconium and titanium,

D represents at least one element selected from phosphorus, tellurium, antimony, tin, cerium, lead, niobium, manganese, arsenic and zinc, and

O represents oxygen

and in which, if a=12,

b is 0.1 to 10,

c is 0.1 to 20,

d is 2 to 20,

e is 0.001 to 10,

f is 0 to 30,

g is 0 to 4 and x has a value determined by the state of oxidation of the other elements.

The catalyst can be introduced per se into the tubes of the shell-and-tube heat exchanger. It can, however, also be applied to inert catalyst supports which are then introduced into the tubes of the shell-and-tube heat exchanger. The excipients used may in this case be conventional porous or non-porous aluminium oxides, silicon dioxide, thorium dioxide, zirconium dioxide, silicon carbide or silicates such as magnesium or aluminium silicate. The support bodies may be of uniform or non-uniform shape, uniformly shaped support bodies having clearly defined surface roughness, for example spheres or hollow cylinders, being preferred.

In general, multi-tube reactors are configured in such a way that a coolant and/or temperature control medium is able to flow in a through-flow direction perpendicularly to the through-flow and orientation of the tubes. The coolant or temperature control medium used is preferably in the form of fluid media. Particularly beneficial is the use of melts of salts such as potassium nitrate, potassium nitrite, sodium nitrite and/or sodium nitrate, or of low-melting metals such as sodium, mercury and alloys of various metals.

According to an advantageous configuration of the device according to the invention, the laminarization means protrudes into the outflow region.

In particular, the protruding laminarization means allows the flow geometry to be changed in the outflow region. It is particularly preferable in this regard for the laminarization means to taper in the direction of the outflow region. The laminarization means is in this case preferably configured as a truncated cone or a cone.

According to a further advantageous configuration of the device according to the invention, the laminarization means comprises a truncated cone.

According to a further advantageous configuration of the device according to the invention, the laminarization means comprises a cone.

The configuration of the laminarization means as a cone or truncated cone, in particular, results in marked laminarization of the flow leading, in turn, to low turbulence rates and, in particular, to a much shorter residence time of the starting materials and products in the individual tubes. This effectively eliminates the risk of autoxidation of the acrolein. In particular, this can also prevent damage to the first reactor by an explosion formed owing to the autoxidation of the acrolein.

Insofar as the laminarization means is a cone or a truncated cone, it is preferred for this cone or truncated cone to have a cone angle in the range of from 15 to 60°, particularly preferably in a range of from 20 to 45° and even more preferably in a range of from 25 to 40°.

The term “cone angle”, as used in the present document, refers in particular to half the opening angle of the cone, i.e. the angle between a projection of the truncated cone or the cone in the region of the tapering and optionally acute region to a plane encompassing the axis of symmetry of the cone or truncated cone.

Furthermore, it is preferred, if the laminarization means is a cone or a truncated cone, for the ratio of the height of the cone or truncated cone (H):diameter of the cone or truncated cone on the side (D) remote from the at least one further reactor to be in a range of from 3:1 to 1:3, particularly preferably in a range of from 2:1 to 1:2 and most preferably in a range of from 1.5:1 to 1:1.5.

The at least one further reactor is preferably also a shell-and-tube heat reactor. However, also conceivable is a reactor, in the reaction chamber of which thermal metal sheets are arranged in such a way as to form between the thermal metal sheets reaction spaces and heat conveyance spaces. Reactors of this type are described, for example, in DE-A-198 48 208 or DE-A-101 08 380.

Like the first reactor, the at least one further reactor also has a catalyst which, insofar as the reactor is a shell-and-tube heat reactor, can be introduced into the tubes of the shell-and-tube heat reactor per se or coated on a catalyst support body. Insofar as the at least one further reactor is a reactor comprising thermal metal sheets arranged in the reaction chamber, the catalyst can be introduced as a feedstock or else coated on the surface of the thermal metal sheets.

The catalyst in the at least one further reactor is preferably a catalyst which catalyzes the conversion of acrolein to acrylic acid. In relation to suitable catalysts in the preparation of acrylic acid from acrolein, reference is again made to “Stets Geforscht”, Volume 2, Chemieforschung im Degussa-Forschungszentrum Wolfgang 1998, pp. 108-126, “Acrolein und Derivate” Chapter, Dietrich Amtz and Ewald Noll, reference being made, in this regard too, to this content as part of the present disclosure.

It is particularly preferred for the catalyst contained in the at least one further reactor to have the composition:

MOₐV_{b}A_{c}B_{d}CₑD_{f}Oₓ

in which

Mo represents molybdenum,

V represents vanadium,

A represents at least one element selected from copper, cobalt, bismuth and iron,

B represents at least one element selected from antinomy, tungsten and niobium,

C represents at least one element selected from silicon, aluminium, zirconium and titanium,

D represents at least one element selected from an alkali metal, an alkaline-earth metal, thallium, phosphorus, tellurium, antimony, tin, cerium, lead, niobium, manganese and zinc and

O represents oxygen

and in which, if a=12,

b is 0.1 to 10,

c is 0.1 to 20,

d is 0.1 to 20,

e is 0.001 to 10,

f is 0 to 30 and x has a value determined by the state of oxidation of the other elements.

A further aspect of the invention proposes a process for the preparation of acrylic acid, wherein:
in a first stage acrolein is produced by gas phase oxidation of propene in a first reactor
and in at least one second stage acrolein is converted into acrylic acid in at least one further reactor,
wherein the first reactor is a multi-tube reactor having a plurality of tubes through which gas is able to flow, which reactor is connected to at least one further reactor via an outflow region,
wherein a gas flow is laminarized in the outflow region using a laminarization means.

Preferred laminarization means are those laminarization means referred to at the outset in relation to the device according to the invention for the preparation of acrylic acid.

The term “laminarization” also refers in the present context, in particular, to a reduction of the Reynolds number. The laminarization may, in particular, be achieved by a corresponding formation of the outflow region. In particular, there may be configured in the outflow region laminarization means which preferably alter the cross section, the shape and/or the length of the outflow region. The process according to the invention may, in particular, be carried out on a device according to the invention.

The Reynolds number may, in particular, be determined based on a flow speed and a viscosity of the flowing gas. The variables required for this purpose can either be measured, for example using a flowmeter, or be calculated based on the known reaction conditions, in particular in view of the temperature, the density of the gases, the mixing ratios of the gases, the state of the catalysts, the density of the catalysts, the catalyst surface area available for a catalytic reaction, the flow cross sections and other factors. In particular, there may in this case be drawn up corresponding Navier-Stokes equations which are solved accordingly. Preferably used in this case are commercial CFD systems such as, for example, the FLUENT™ software package. The details and advantages disclosed for the device for the preparation of acrylic acid are similarly applicable and transferable to the process according to the invention for the preparation of acrylic acid and vice versa.

Preferably, the process according to the invention includes as a further process step, in addition to the production of the acrolein and the conversion of the acrolein to form acrylic acid, the purification of the acrylic acid thus obtained by distillation, crystallization, extraction or by a combination of these purification processes.

The purification is preferably carried out in such a way that first of all the product gas mixture obtained is subjected to total condensation in what is known as a quench tower in water so as to obtain an aqueous acrylic acid solution. However, it is also conceivable to absorb the acrylic acid in high-boiling solvents such as, for example, a mixture of 75% by weight diphenyl ether and 25% by weight diphenyl. Absorption of the acrylic acid is usually followed by further purification by distillation wherein, in the case of aqueous acrylic acid solutions as the starting composition, azeotrope distillation is frequently carried out in the presence of suitable entraining agents such as toluene, a crude acrylic acid being retained as a bottom product. If the reaction gas mixture was absorbed in high-boiling solvents, a crude acrylic acid is usually drawn off in the side stream of a rectification device.

The crude acrylic acid thus obtained can then be further distilled for further purification in order, in particular, to separate low-boiling substances. The crude acrylic acid can also be further purified by crystallization, in particular by suspension crystallization, there ultimately being obtained a pure acrylic acid having an acrylic acid content of at least 99% by weight.

A further aspect of the invention proposes a process for the preparation of a hydrophilic polymer, wherein the preferably purified acrylic acid obtainable by the process according to the invention is radically polymerized. Preferably, the polymerization is carried out in such a way that the preferably purified acrylic acid obtainable by the process according to the invention is radically polymerized in partially neutralized form in an aqueous solution in the presence of crosslinking agents so as to form a hydrogel, the hydrogel subsequently being size-reduced and dried and the polymer particles thus obtained subsequently being surface-modified, preferably surface-post-crosslinked. What are known as superabsorbers are thus obtained. Further details concerning superabsorbers, in particular concerning the preparation thereof, are disclosed in “Modern Superabsorbent Polymer Technology”, F L Buchholz, A T Graham, Wiley-VCH, 1998.

A further aspect of the present invention is formed by a method for the production of a water-absorbent hygiene article in which a hydrophilic, preferably water-absorbent, polymer, particularly preferably a superabsorber, prepared by the foregoing process, is incorporated into at least one hygiene article component. A hygiene article component of this type is preferably the core of a diaper or sanitary towel.

The present invention also relates to chemical products, such as fibers, shaped articles, films, foams, superabsorbent polymers, detergents, special polymers for the fields of waste water treatment, emulsion paints, cosmetics, textiles, leather dressing or paper production or hygiene articles, which are at least based on or contain purified acrylic acid, the purified acrylic acid being obtainable by the aforementioned process.

Finally, there is also proposed a use of preferably purified acrylic acid obtainable by the process according to the invention for the preparation of acrylic acid, in or for the production of fibers, shaped articles, films, foams, superabsorbent polymers or hygiene articles, detergents or special polymers for the fields of waste water treatment, emulsion paints, cosmetics, textiles, leather dressing or paper production.

The invention will be described hereinafter in greater detail with reference to the appended figures without thereby entailing any limitation to the embodiments, advantages and details shown therein. In the drawings:

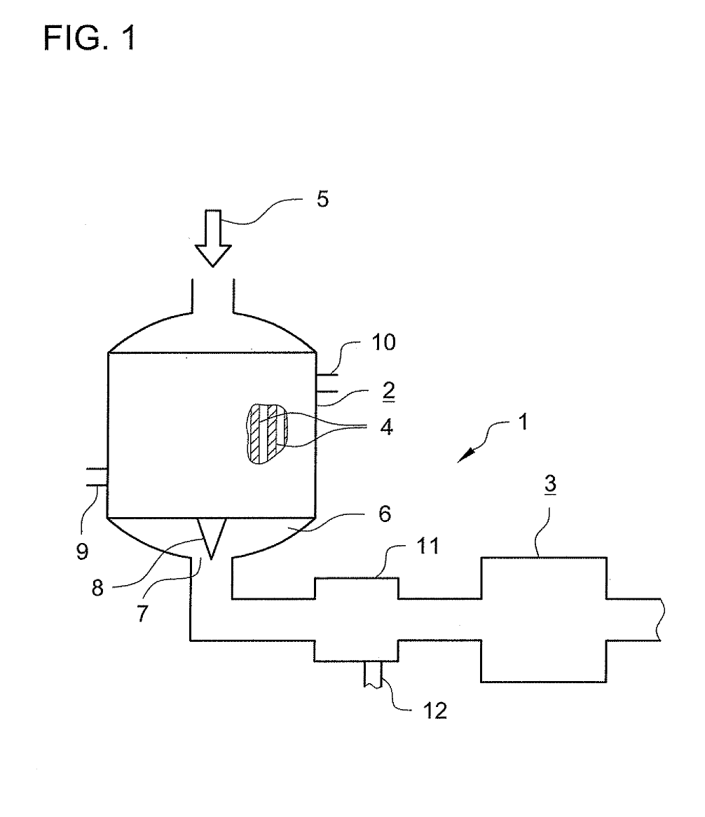

FIG. 1 shows schematically in cross section a device according to the invention;

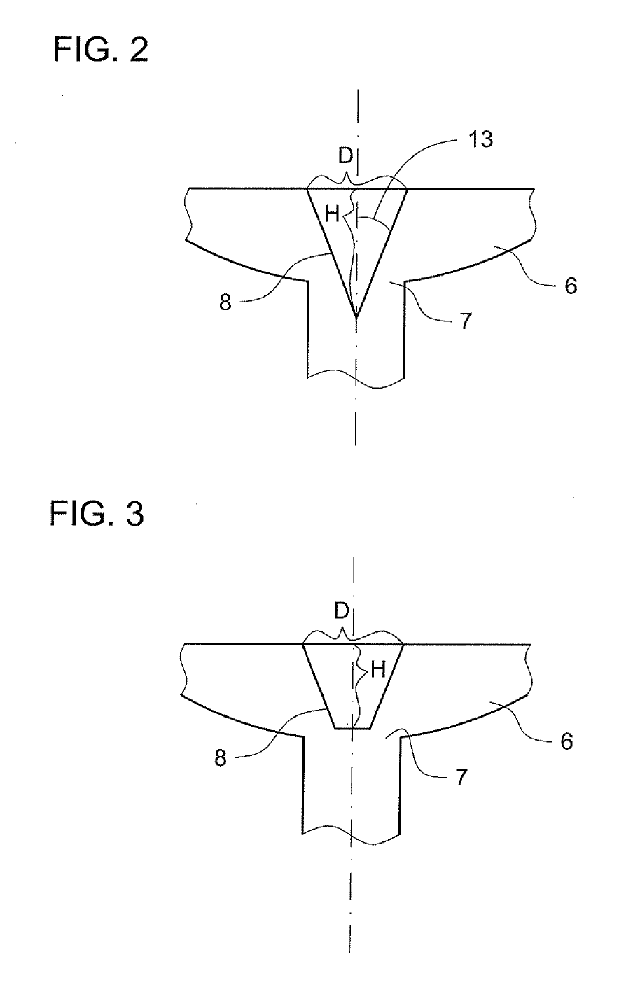

FIG. 2 shows schematically a detail of a device according to the invention; and

FIG. 3 shows schematically a detail of a further embodiment of a device according to the invention.

FIG. 1 shows schematically a device 1 according to the invention for the preparation of acrylic acid. The device 1 comprises a first reactor 2 and at least one further reactor 3. At least the first reactor 2 is a multi-tube reactor comprising a plurality of tubes 4, merely a detail of which is illustrated, by way of example, for the sake of clarity. The tubes 4 may be arranged as a ring of tubes in order to achieve better circulation with a coolant, preferably a salt melt. Each of the tubes 4 comprises a catalyst for the catalytic reaction of propene with oxygen to form acrolein. In particular, this catalyst may be a multi-component catalyst based on bismuth molybdate. A first gas flow 5 may flow through the tubes 4. The first gas flow 5 comprises the starting materials of the gas phase oxidation, i.e. propene and oxygen. The gas leaving the tubes 4 is combined in a collection chamber 6. This collection chamber 6 is connected to at least one further reactor 3 via an outflow region 7. The outflow region 7 comprises a laminarization means 8 causing laminarization of a flow profile of a gas flowing through the outflow region 7. In this case, the laminarization means 8 protrudes into the outflow region 7 and tapers in the direction of the outflow region 7. If the tubes 4 are arranged as a ring of tubes, the laminarization means is preferably arranged below the tube-free internal region of the ring of tubes. In the present first embodiment, the laminarization means 8 is configured in the form of a cone. The term “flow laminarization” refers to a reduction of the Reynolds number of this flow. The flow laminarization increases the average flow speed and reduces the residence time of the starting materials and products in the tubes 4, thus substantially preventing autoxidation of the mixture of gases. As the reactions taking place in the tubes 4 include exothermic reactions, it is advantageous to adjust the temperature of the tubes. There is configured for this purpose a coolant inlet 9 and a coolant outlet 10 through which a coolant is able to flow through the first reactor 2 and around the tubes 4. The coolant inlet 9 and coolant outlet 10 may be connected to corresponding means for conveying a coolant, for example a corresponding pump (not shown).

The mixture of product gases leaves the first reactor 2 via the outflow region 7. In a mixer 11, the acrolein is mixed with oxygen which may be added via a feed line 12. In the further reactor 3, the acrolein is then oxidized to form acrylic acid.

FIG. 2 shows schematically a detail of a device 1 according to the invention. Shown are outflow region 7, the collection chamber 6 and the laminarization means 8. The laminarization means 8 comprises a cone having a cone angle 13, having a height H and having a diameter D on the side remote from the at least one further reactor.

FIG. 3 shows schematically a detail of a further embodiment of a device 1 according to the invention. Shown, in this case too, are the collection chamber 6, the outflow region 7 and the laminarization means 8. The laminarization means 8 is configured as a truncated cone.

### LIST OF REFERENCE NUMERALS

1 Device for the preparation of acrylic acid
2 First reactor
3 Further reactor
4 Tubes
5 First gas flow
6 Collection chamber
7 Outflow region
8 Laminarization means
9 Coolant inlet
10 Coolant outlet
11 Mixer
12 Feed line
13 Cone angle
D Diameter of the cone or the truncated cone on the side remote from the at least one further reactor
H Height of the cone or the truncated cone

1. The device for the preparation of acrylic acid, comprising:
a first reactor and
at least one further reactor,
wherein at least the first reactor is a multi-tube reactor having a plurality of tubes comprising a catalyst which catalyses the synthesis of acrolein,
wherein said tubes open into a collection chamber which can be fluidically connected to the at least one further reactor via an outflow region,
wherein the said outflow region comprises a laminarization means causing laminarization of a flow profile of a gas flowing through said outflow region.

2. The device according to claim 1, wherein said laminarization means protrudes into said outflow region.

3. The device according to claim 1, claim 2, wherein said laminarization means tapers in the direction of said outflow region.

4. The device according to claim 1, wherein the laminarization means comprises a truncated cone.

5. The device according to claim 3, wherein the laminarization means comprises a cone.

6. The device according to claim 4, wherein or said truncated cone has a cone angle from about 15 to about 60°.

7. The device according to claim 4, wherein the ratio of the height of said truncated cone:diameter of the truncated cone on the side remote from the at least one further reactor is in a range of from about 3:1 to about 1:3.

8. The process for the preparation of acrylic acid, wherein:
in a first stage acrolein is produced by gas phase oxidation of propene in a first reactor,
and in at least one second stage acrolein is converted into acrylic acid in at least one further reactor,
wherein said first reactor is a multi-tube reactor having a plurality of tubes through which gas is able to flow, wherein said first reactor is connected to at least one further reactor via an outflow region,
wherein a gas flow is laminarized in the said outflow region using a laminarization means.

9. The process according to claim 8, wherein the said process includes as a further process step the purification of said acrylic acid by distillation, crystallization, extraction or by a combination of these purification processes.

10. The process according to claim 8, wherein said process is carried out in a device comprising:
a first reactor and
at least one further reactor,
wherein said first reactor is a multi-tube reactor having a plurality of tubes comprising a catalyst which catalyses the synthesis of acrolein,
wherein said tubes open into a collection chamber which can be fluidically connected to the at least one further reactor via an outflow region,
wherein said outflow region comprises a laminarization means causing laminarization of a flow profile of a gas flowing through said outflow region.

11. The acrylic acid obtainable produced according to the process of claim 8.

12. The process for the preparation of a hydrophilic polymer, wherein the acrylic acid produced according to claim 9 is radically polymerized.

13. The hydrophilic polymer obtainable using a process according to claim 12.

14. The method for the production of a water-absorbent hygiene article, wherein a hydrophilic polymer according to claim 13 is combined with at least one hygiene article component.

15. An article selected from the group consisting of fibers, shaped articles, films, foams, superabsorbent polymers, detergents, special polymers for the fields of waste water treatment, emulsion paints, cosmetics, textiles, leather dressings or paper production or hygiene articles, wherein said article is based on the acrylic acid produced in accordance with claim 9.

16. A method of using an acrylic acid produced according to claim 9 in or for the production of an article selected from the group consisting of fibers, shaped articles, films, foams, superabsorbent polymers or hygiene articles, detergents or special polymers for the fields of waste water treatment, emulsion paints, cosmetics, textiles, leather dressings or paper production.

17. The device according to claim 2, wherein said laminarization means tapers in the direction of said outflow region.

18. The device according to claim 2, wherein the laminarization means comprises a truncated cone.

19. The device according to claim 4, wherein the laminarization means comprises a cone.

20. The device according to claim 5, wherein said cone has a cone angle from about 15 to about 60°.

21. The device according to claim 18, wherein said truncated cone has a cone angle from about 15 to about 60°.

22. The device according to claim 5, wherein the ratio of the height of said cone:diameter of the cone on the side remote from the at least one further reactor is from about 3:1 to about 1:3.

23. The device according to claim 6, wherein the ratio of the height of said truncated cone:diameter of the truncated cone on the side remote from the at least one further reactor is from about 3:1 to about 1:3.

24. The process according to claim 10, wherein said laminarization means of said device protrudes into said outflow region.

25. The process according to claim 10, wherein said laminarization means tapers in the direction of said outflow region.

26. The process according to claim 24, wherein said laminarization means tapers in the direction of said outflow region.

27. The process according to claim 10, wherein said laminarization means comprises a truncated cone.

28. The process according to claim 24, wherein said laminarization means comprises a truncated cone.

29. The process according to claim 25, wherein said laminarization means comprises a cone.

30. The process according to claim 27, wherein said truncated cone has a cone angle of from about 15 to about 60°.

31. The process according to claim 27, wherein the ration of the height of said truncated cone:diameter of the truncated cone on the side remote from the at least one further reactor is from about 3:1 to about 1:3.
